(19) United States
(12) Patent Application Publication
(10) Pub. No.: US 2012/0097157 A1
(43) Pub. Date: Apr. 26, 2012

(54) APPARATUS, SYSTEMS, AND METHODS FOR SECURING A BREATHING TUBE TO AN ARM

(75) Inventor: FELINO V. CORTEZ, JR., Bowie, MD (US)

(73) Assignee: Vapotherm, Inc., Stevensville, MD (US)

(21) Appl. No.: 12/910,155

(22) Filed: Oct. 22, 2010

(51) Int. Cl. A61M 16/08

(52) U.S. Cl. 128/202.13

(57) ABSTRACT

Methods, systems, and apparatus for securing a breathing tube to a user are disclosed. An exemplary apparatus is an armband including a strap sized to encircle the arm of the user, a connecting element coupled to the strap to secure the strap around the arm, and at least one loop coupled to the strap, the loop receiving the breathing tube and securing the breathing tube to the arm. In use, the armband may secure a breathing tube to a user by encircling the arm of the user with the strap, securing the strap around the arm with the connecting element, and attaching the breathing tube to the arm. The armband may be used in a system for assisting a user with the work of breathing that includes a source of breathing air, a breathing tube, and a patient interface.

### FIELD OF THE INVENTION

The present invention relates generally to assisted breathing systems, and more particularly to apparatus, systems, and methods for securing a breathing tube to an arm.

### BACKGROUND OF THE INVENTION

Systems for assisting with the work of breathing are widely used for treating patients with respiratory ailments. Such breathing systems generally include a source of air that is delivered to a patient via a breathing tube. The end of the breathing tube may include a patient interface (such as a breathing mask) to provide air to the patient.

Assisted breathing systems are often used when a patient is sleeping. For example, nasal continuous positive airway pressure (CPAP) is a common treatment for patients suffering from sleep apnea. An important factor in effective CPAP therapy is ensuring that there is no discomfort or leakage between the patient and the patient interface. However, these problems may arise due to a patient's movement while asleep. Accordingly, improvements are desired for effective assisted breathing treatment.

### SUMMARY OF THE INVENTION

The present invention is embodied in methods, systems, and apparatus for securing a breathing tube to a user.

An exemplary apparatus is directed to an armband for securing a breathing tube to a user. The armband includes a strap sized to encircle the arm of the user, a connecting element coupled to the strap to secure the strap around the arm of the user, and at least one loop coupled to the strap, the loop configured to receive the breathing tube and secure the breathing tube to the arm of the user.

An exemplary system is directed to a system for assisting a user with the work of breathing. The system includes a source of breathing air, a breathing tube having a first end and a second end with the first end coupled to the source of breathing air, a patient interface coupled to the second end of the breathing tube to provide the breathing air to the user, and the armband described above.

An exemplary method is directed to securing a breathing tube to a user. The method includes encircling the arm of the user with a strap, securing the strap around the arm of the user with a connecting element, and attaching the breathing tube to the arm of the user.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that according to common practice, the various features of the drawings are not drawn to scale unless otherwise indicated. On the contrary, the dimensions of the various features may be expanded or reduced for clarity. Included in the drawings are the following figures:

FIG. 1 illustrates of an exemplary armband for securing a breathing tube to a user in accordance with aspects of the present invention;

FIG. 2 depicts the armband of FIG. 1 in an unsecured position;

FIG. 3 depicts the armband of FIG. 1 without a secured breathing tube;

FIG. 4 is a diagram of the armband of FIG. 3;

FIG. 5 depicts an exemplary strap of the armband of FIG. 1;

FIG. 6 is a diagram of the strap of FIG. 5;

FIG. 7 depicts an exemplary loop of the armband of FIG. 1;

FIG. 8 depicts the exemplary loop of FIG. 7 with a secured breathing tube;

FIGS. 9A and 9B depict a top view and a side view, respectively, of the exemplary loop of FIG. 7;

FIG. 10 depicts an exemplary system for assisting a user with the work of breathing in accordance with aspects of the present invention; and

FIG. 11 depicts is a flowchart illustrating an exemplary method for securing a breathing tube to a user in accordance with aspects of the present invention.

### DETAILED DESCRIPTION OF THE INVENTION

The exemplary apparatus, systems, and methods disclosed herein are broadly usable with systems designed to assist a patient with the work of breathing. Appropriate assisted breathing systems include a breathing tube configured to deliver breathing gas to a patient. The exemplary embodiments disclosed herein are particularly suitable for use with continuous positive airway pressure (CPAP) breathing systems. Suitable CPAP systems for use with the present invention will be known to one of ordinary skill in the art from the description herein.

The exemplary apparatus, systems, and methods disclosed herein are also broadly usable in conjunction with respiratory treatment for patients at any time. However, the exemplary embodiments of the invention are particularly suitable for use with patients during sleep. As set forth herein, the disclosed apparatus, systems, and methods may enable sleeping patients to comfortably and properly receive respiratory treatment despite movements during sleep. Suitable respiratory treatments that will benefit from utilizing aspects of the present invention will be known to one of ordinary skill in the art from the description herein.

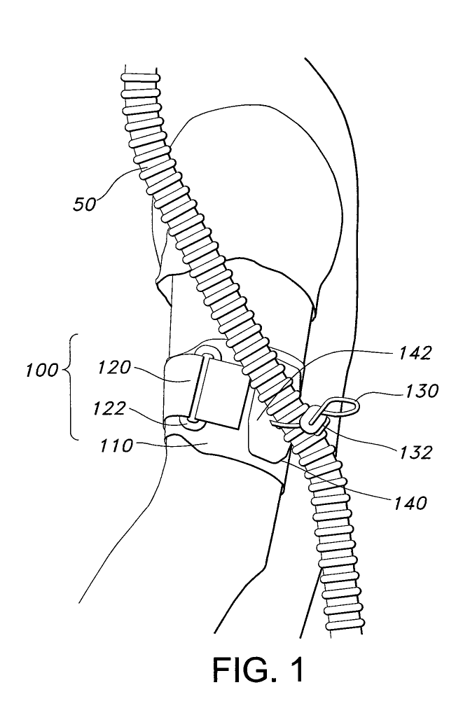

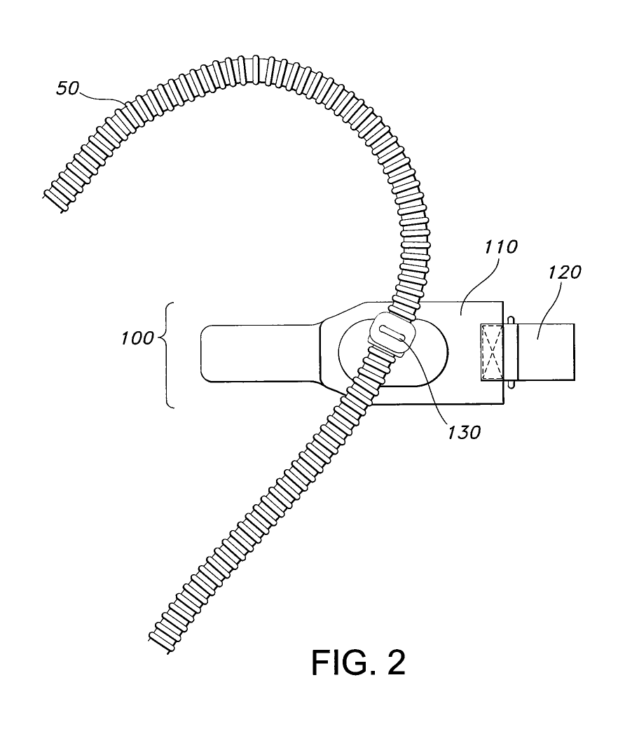

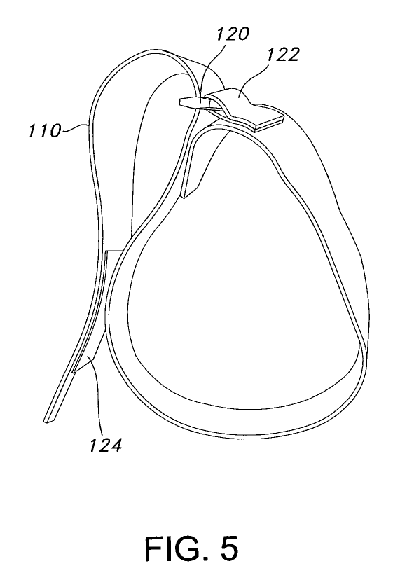

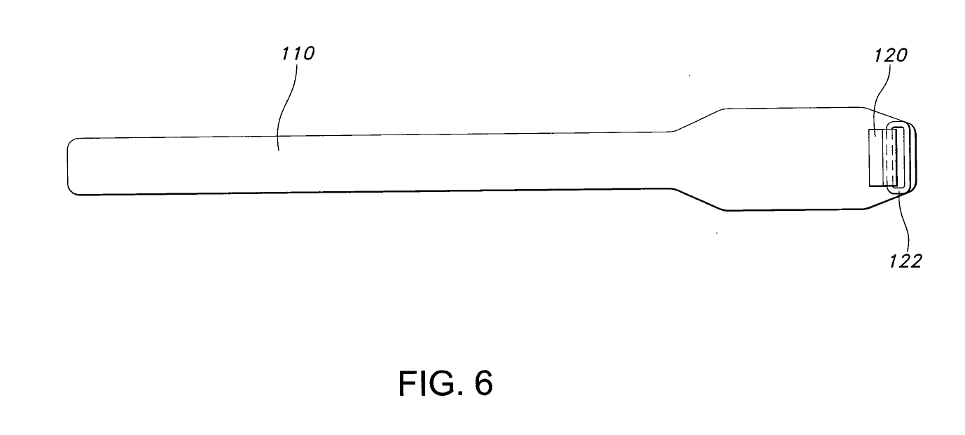

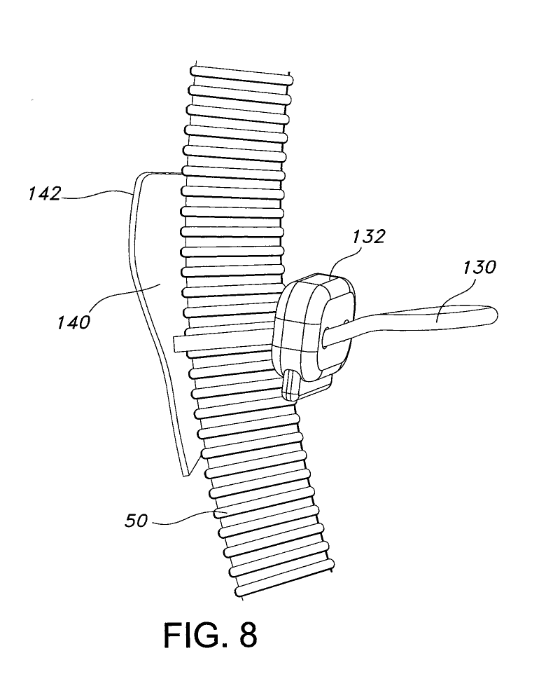

Referring now to the drawings, FIGS. 1-9B illustrate an exemplary armband 100 for securing a breathing tube to a user in accordance with aspects of the present invention. Armband 100 is suitable for assisting in the provision of respiratory therapy to a user. To this end, armband 100 is usable in conjunction with a breathing tube 50, as illustrated in FIGS. 1 and 2. Generally speaking, armband 100 includes a strap 110, a connecting element 120, and at least one loop 130. Additional details of armband 100 are provided herein.

Strap 110 is configured to encircle the arm of a user. As shown in FIGS. 1 and 3-6, strap 110 is long enough to wrap all of the way around the upper arm of a user. Strap 110 may be formed from flexible fabric or other similar material that is stretchable or elastic. Suitable materials for strap 110 include, for example, rubber, elastomeric material, a natural fiber such as cotton or wool, a natural fiber blend, nylon, polypropylene, Velcro, breathe-o-prene, felt, foam, etc. Other suitable materials will be understood by one of skill in the art from the description herein.

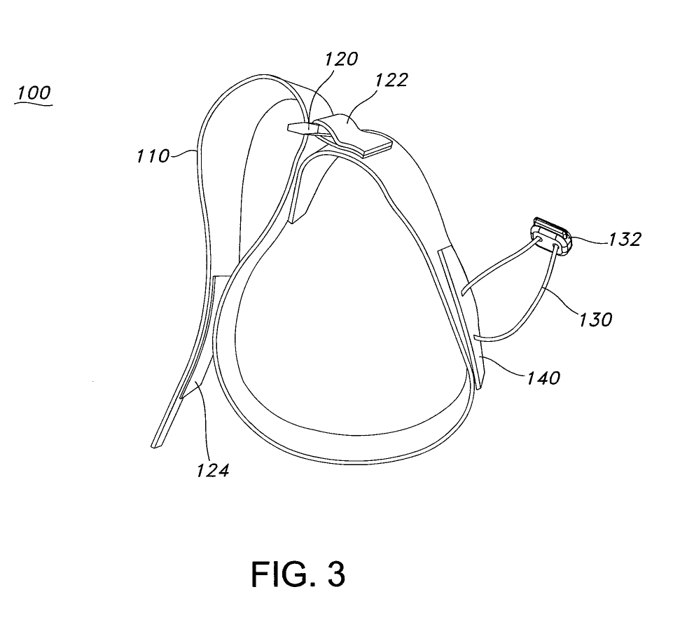

Connecting element 120 is coupled to strap 110. Connecting element 120 secures strap 110 in place around the arm of a user. In an exemplary embodiment, connecting element 120 includes a buckle 122 having a slot for receiving strap 110, as shown in FIGS. 3-6. The end of strap 110 may be passed through buckle 122 and cinched back on itself to secure the armband 100 in place on the user's arm.

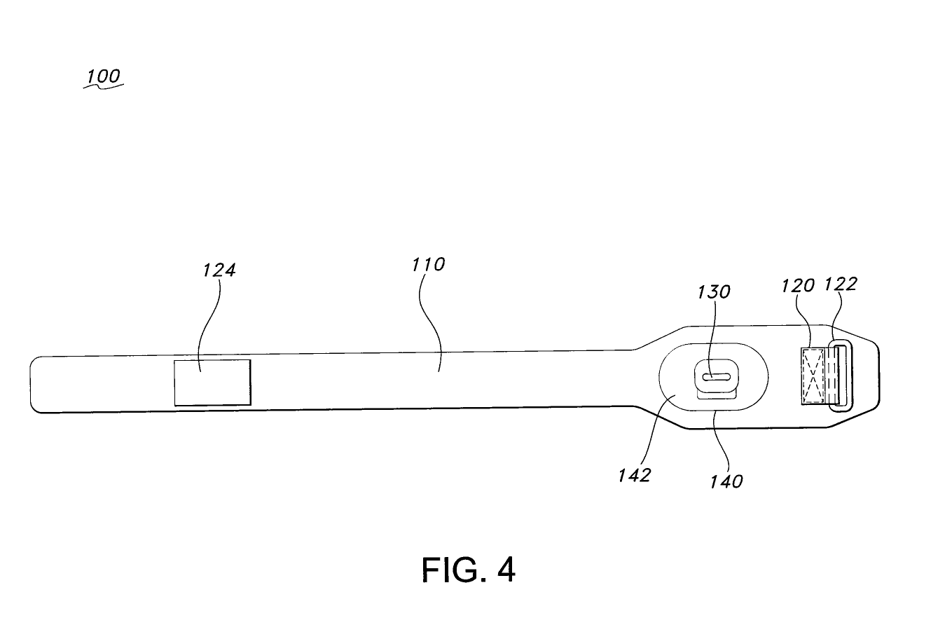

While connecting element 120 is illustrated as a buckle 122, it is not so limited. In alternative embodiments, connecting element 120 may comprise buttons, snaps, hook-and-loop fasteners, a ratchet, or any other suitable structures adapted to secure strap 110 in place on the user's arm. In one embodiment, connecting element 120 may be an inflatable system that secures strap 110 in place through the inflation of one or more bladders located within the strap 110 or on a surface of the strap 110 adjacent the arm of the user. Further, connecting element 120 may comprise more than one of the above-mentioned connecting elements to provide a reliable connection. For example, as shown in FIG. 4, connecting element 120 may comprise a buckle 122 in combination with a hook or loop fastener 124. The end of strap 110 may be cinched back on itself through buckle 122 and affixed to another portion of strap 110 having a loop or hook fastener (not shown) corresponding to the hook or loop fastener 124, thereby securing armband 100 in place on the user's arm.

It is further contemplated that strap 110 may comprise elastic material. Accordingly, strap 110 may be securable around the arm of the user by elastic contraction of the strap itself. In this embodiment, connecting element 120 may be omitted from armband 100.

At least one loop 130 is coupled to strap 110. As shown in FIGS. 3 and 7-9, loop 130 is configured to receive breathing tube 50 and secure breathing tube 50 to the arm of the user. Loop 130 may be formed from a flexible or elastic cord, rope, band, or other similar material. Suitable materials for loop 130 include, for example, nylon.

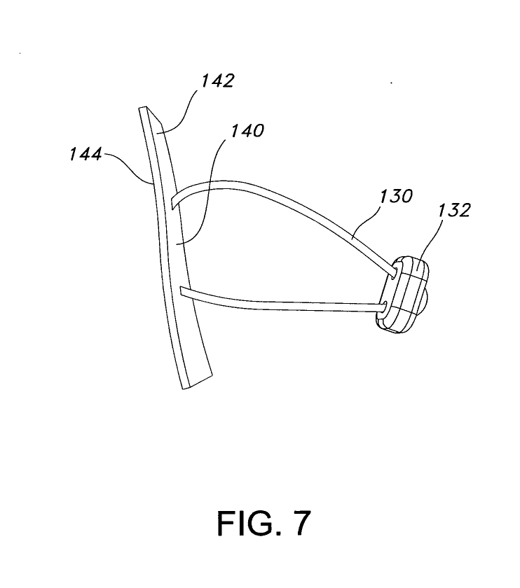

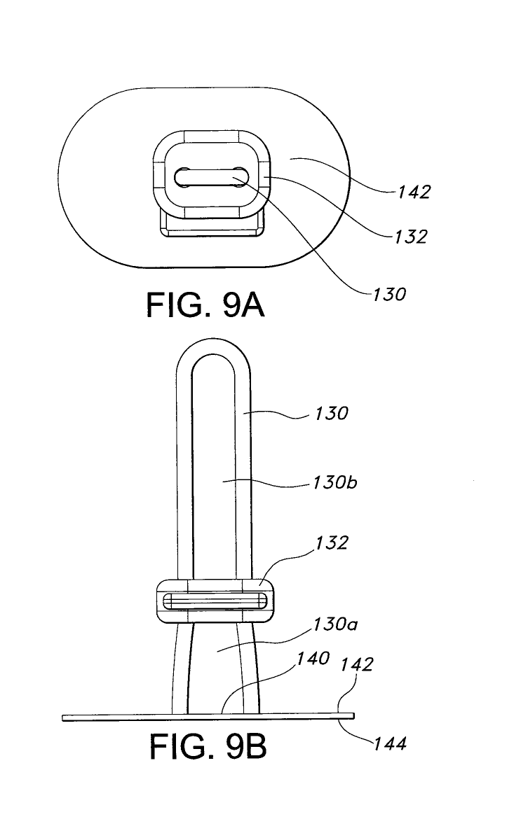

In an exemplary embodiment, loop 130 comprises a flexible drawstring including a clasp 132, as shown in FIGS. 7-9. The illustrated clasp 132 separates loop 130 into two sub loops 130a and 130b (FIG. 9B). Clasp 132 may be adjustable by a user to control the size of the sub loops 130a/130b in order to secure the tube 50 within the respective sub loop. For example, if the tube 50 is positioned within sub loop 130a a user may slide clasp 132 toward fastener 140 to decrease the size of the opening of sub loop 130a, thereby preventing breathing tube 50 from moving when breathing tube 50 is received within sub loop 130a. On the other hand, if the tube 50 is positioned within sub loop 130b a user may slide clasp 132 away from fastener 140 to decrease the size of the opening of sub loop 130b, thereby preventing breathing tube 50 from moving when breathing tube 50 is received within sub loop 130b. In particular, it may be desirable that sub loops 130a, b prevent movement of breathing tube 50 in the axial direction, i.e., sliding forward or backward through loop 130, when tube 50 is received by a respective sub loop. This may prevent breathing tube 50 from transmitting force to a patient interface, as outlined in more detail below. Positioning breathing tube 50 within sub loop 130a allows the tube to be secured adjacent to the user's arm. On the other hand, positioning breathing tube 50 within sub loop 130b allows the tube to be secured near the user's arm while allowing some movement of the tube due to the portion of loop 130 positioned between the clasp 132 and the fastener 140. Thus, a user is able to customize the armband to provide greater comfort to the user.

While the at least one loop 130 may be integrally formed or sewn into the strap, it is desirable that the at least one loop 130 be releasably coupled to strap 110. To this end, a fastener 140 may be coupled to the loop 130 that is configured to releasably couple loop 130 to strap 110. In an exemplary embodiment, fastener 140 comprises a patch 142 including a hook or loop connector 144, as shown in FIGS. 3 and 7-9. The patch 142 may be affixed to a portion of strap 110 having a loop or hook connector (not shown) corresponding to the hook or loop connector 144, thereby coupling loop 130 to strap 110. By releasably coupling loop 130 to strap 110, a user of armband 100 may be enabled to leave the proximity of the breathing tube 50 (and the associated assisted breathing system) without having to first remove breathing tube 50 from loop 130. This may be particularly useful when the user must quickly or briefly leave his or her bed (or the vicinity of the assisted breathing system) during respiratory treatment.

While fastener 140 is illustrated as a patch including a hook and/or loop connector 144, it is not so limited. In alternative embodiments, fastener 140 may comprise buttons, snaps, and/or any other suitable fasteners adapted to releasably couple loop 130 to strap 110.

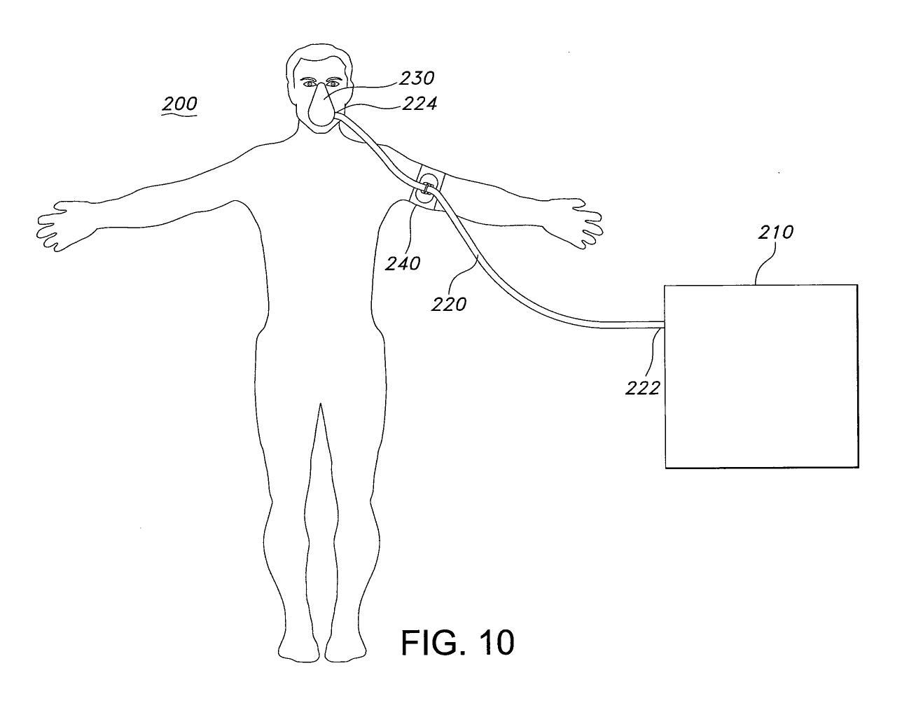

FIG. 10 illustrates an exemplary system 200 for assisting a user with the work of breathing in accordance with aspects of the present invention. System 200 is usable to provide respiratory therapy to a user. Generally speaking, system 200 includes a source of breathing air 210, a breathing tube 220, a patient interface 230, and an armband 240. Additional details of system 200 are provided herein.

Source 210 provides breathing air for a patient. In an exemplary embodiment, breathing air source 210 is a conventional continuous positive airway pressure (CPAP) machine. Other suitable sources of breathing air 210 will be known to one of ordinary skill in the art from the description herein.

Breathing tube 220 delivers air from the breathing air source 210 to the patient. Breathing tube 220 has a first end 222 that is coupled to the source of breathing air 210 and a second end 224. Breathing air produced by source 210 is received into the first end 222 of breathing tube 220. The breathing air may be transmitted through breathing tube 220, for example, by pressure applied by breathing air source 210 and/or by inhalation by the patient. Suitable breathing tubes will be known to one of ordinary skill in the art from the description herein.

Patient interface 230 is coupled to the second end 224 of breathing tube 220. Patient interface 230 is configured to provide the breathing air transmitted through breathing tube 220 to the user. Patient interface 230 may be disconnected from breathing tube 220 at the second end 224 to allow the user to quickly leave the bed and breathing device, for example, without removing the patient interface 230. Additionally, patient interface 230 may be easily connectable to the breathing tube 220 to facilitate reconnection, e.g., upon return to the bed and breathing device. In an exemplary embodiment, patient interface 230 is a CPAP breathing mask. Alternatively, patient interface 230 may be a nasal cannula. Other suitable patient interfaces will be known to one of ordinary skill in the art from the description herein.

Armband 240 encircles the arm of the user of system 200. Armband 240 is an armband substantially as described above with respect to armband 100. Armband 240 is adapted to receive the breathing tube 220 and secure the breathing tube 220 to the arm of the user. As described above, armband 240 includes a loop configured to receive breathing tube 220 and secure breathing tube 220 in place adjacent the user's arm. The loop may be adjustable in size and releasably coupled to armband 240, substantially as described above.

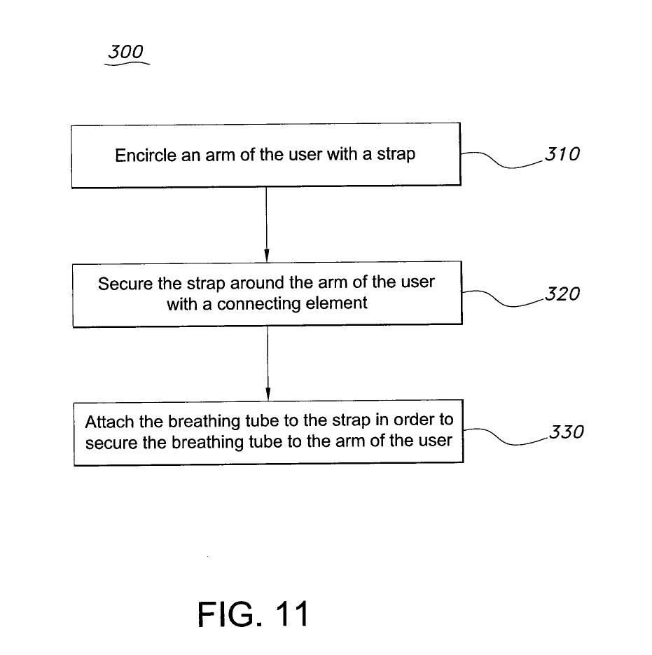

FIG. 11 is a flowchart illustrating an exemplary method 300 for securing a breathing tube to a user in accordance with aspects of the present invention. Method 300 is suitable for assisting in the provision of respiratory therapy to a user. Generally speaking, method 300 includes encircling an arm of a user with a strap, securing the strap around the arm of the user, and attaching the breathing tube to the strap. Additional details of method 300 are provided herein. For the purposes of illustration, the steps of method 300 are described herein with reference to the exemplary armband 100 described above.

In step 310, a strap is encircled around the arm of a user. In an exemplary embodiment, strap 110 is encircled around the arm of the user. The user may personally encircle strap 110 around his or her arm, or a care provider may encircle strap 110 around the user's arm. Armband 100 is desirably placed around the user's upper arm, in order to be proximate the user's airways, which, in turn may be used to positions the breathing tube 50/220 proximate the user's airways. Additionally, armband 100 is desirably placed around the arm of the user closest to the source of breathing air.

In step 320, the strap is secured around the user's arm with a connecting element. In an exemplary embodiment, strap 110 is secured to the arm of the user with connecting element 120. As stated above, the user may personally secure strap 110 to his or her arm, or a care provider may secure strap 110 to the user's arm with connecting element 120.

In step 330, the breathing tube is attached to the strap. In an exemplary embodiment, breathing tube 50 is passed through the at least one loop 130. Loop 130 may be coupled to strap 110 in order to secure breathing tube 50 to the user's arm Where loop 130 is releasably coupled as described above, loop 130 may be coupled to strap 110 before or after breathing tube 50 is passed through loop 130.

It will be understood to one of ordinary skill in the art that method 300 is not limited to the above steps. Method 300 may include additional or alternative steps. Examples of such steps are outlined below.

Method 300 may further comprise the step of adjusting a size of the at least one loop to prevent movement of the breathing tube within the loop. In an exemplary embodiment, a size of loop 130 may be adjusted by sliding clasp 132. For example, the user or a care provider may slide clasp 132 to decrease the size of the opening of loop 130, thereby preventing breathing tube 50 from moving when breathing tube 50 is received within loop 130. When breathing tube 50 is so secured, it may be desirable to allow breathing tube 50 a measure of slack between the armband 100 and the user's airways. This may be important to accommodate any relative movement between the user's face and arm during respiratory treatment without pulling breathing tube 50 away from the user's face.

Additionally, method 300 may comprise the step of detaching the at least one loop from the strap. In an exemplary embodiment, the at least one loop 130 may be releasably coupled to strap 110 with fastener 140. The user or a care provider may detach loop 130 from strap 110 by disconnecting the fastener 140 from the strap 110. As stated above, this may be particularly useful when the user must quickly or briefly leave his or her bed (or the vicinity of the assisted breathing system) during respiratory treatment.

The exemplary apparatus, systems, and methods described herein may provide advantages over conventional assisted breathing systems as set forth below. In conventional assisted breathing systems, the attachment of the breathing tube to the source of breathing air may cause a pulling or tugging force to be exerted on the patient interface. This may lead to discomfort or leaks between the patient interface and the user. Through using the armband of the present invention, the pulling or tugging force may be prevented or displaced to the user's arm, while a comfortable, leak-free engagement is maintained between the user's face and the patient interface.

Further, the exemplary apparatus, systems, and methods described herein may be particularly advantageous for patients receiving respiratory therapy during sleep. In general, a patient may move or change positions in bed, which can generate a pulling or tugging force on the patient interface. By employing the armband of the present invention, the patient may be free to move normally during sleep, without forces being exerted upon the patient interface.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. An armband for securing a breathing tube to a user, the armband comprising:
a strap sized to encircle an arm of the user;
a connecting element coupled to the strap to secure the strap around the arm of the user; and
at least one loop coupled to the strap, the at least one loop configured to receive the breathing tube and secure the breathing tube to the arm of the user.

2. The armband of claim 1, wherein the at least one loop is adjustable in size to prevent movement of the breathing tube within the loop.

3. The armband of claim 1, wherein the at least one loop is releasably coupled to the strap.

4. The armband of claim 3, further comprising:
a fastener coupled to the at least one loop, the fastener configured to releasably couple the at least one loop to the strap.

5. The armband of claim 4, wherein the fastener comprises a hook or loop fastener.

6. A system for assisting a user with the work of breathing, the system comprising:
a source of breathing air;
a breathing tube having a first end and a second end, the first end coupled to the source of breathing air to receive the breathing air;
a patient interface coupled to the second end of the breathing tube to provide the breathing air to the user; and
the armband of claim 1.

7. The system of claim 6, wherein the patient interface is one of a breathing mask and a nasal cannula.

8. The system of claim 6, wherein the armband comprises at least one loop configured to receive the breathing tube and secure the breathing tube to the arm of the user.

9. The system of claim 8, wherein the at least one loop is adjustable in size to prevent movement of the breathing tube within the loop.

10. The system of claim 8, wherein:
the armband further comprises a strap configured to encircle the arm of the user; and
the at least one loop is releasably coupled to the strap.

11. The system of claim 10, wherein the armband further comprises a fastener coupled to the at least one loop, the fastener configured to releasably couple the at least one loop to the strap.

12. The system of claim 11, wherein the fastener comprises a hook or loop fastener.

13. A method for securing a breathing tube to a user, the method comprising the steps of:
encircling an arm of the user with a strap;
securing the strap around the arm of the user with a connecting element; and
attaching the breathing tube to the strap in order to secure the breathing tube to the arm of the user.

14. The method of claim 13, wherein the attaching step comprises:
passing the breathing tube through at least one loop; and
coupling the at least one loop to the strap.

15. The method of claim 13, wherein the attaching step comprises:
coupling at least one loop to the strap; and
passing the breathing tube through the at least one loop.

16. The method of claim 13, wherein at least one loop is coupled to the strap, and wherein the attaching step comprises:
passing the breathing tube through the at least one loop coupled to the strap.

17. The method of claim 16, further comprising the step of adjusting a size of the at least one loop to prevent movement of the breathing tube within the loop.

18. The method of claim 17, further comprising detaching the at least one loop from the strap.
